(19) United States
(12) Patent Application Publication
(10) Pub. No.: US 2025/0369971 A1
(43) Pub. Date: Dec. 4, 2025

(54) APPARATUS FOR MEASURING KINASE ACTIVITY WITH A FUNCTION OF DETERMINING THE NUMBER OF EFFECTIVE PHOSPHORYLATION SITES AND METHOD THEREOF

(71) Applicant: CANON MEDICAL SYSTEMS CORPORATION, Otawara-shi (JP)

(72) Inventor: Hiroto SATAKE, Boston, MA (US)

(73) Assignee: CANON MEDICAL SYSTEMS CORPORATION, Otawara-shi (JP)

(21) Appl. No.: 19/223,661

(22) Filed: May 30, 2025

(30) Foreign Application Priority Data
May 31, 2024 (JP) 2024-089534

(51) Int. Cl. G01N 33/573; G01N 33/543; G01N 33/574

(52) U.S. Cl. CPC G01N 33/573; G01N 33/54386; G01N 33/57496; G01N 2440/14

(57) ABSTRACT

A method for measuring kinase activity taking into consideration the number of phosphorylation sites. The method for measuring kinase activity includes making the number of effective phosphorylation sites correspond to a substrate protein; acquiring the amount of the substrate protein on a base; and estimating the kinase activity based on the number of effective phosphorylation sites corresponding to the substrate protein on the base and the acquired amount of the substrate protein on the base.

### TECHNICAL FIELD

The embodiments disclosed in the present description and the drawings relate to an apparatus for measuring kinase activity with a function for determining the number of effective phosphorylation sites and method thereof.

### BACKGROUND ART

Information-transmitting molecular mechanisms exist within cells to express various functions, and post-translational modifications of proteins, especially protein phosphorylation, play an important role in signal transduction. Intracellular signal transduction mechanisms form complex networks connecting numerous molecules together, and the abnormal signal transduction caused by activation through mutation or overexpression of a protein kinase of a protein phosphorylation enzyme is conceivably involved in many diseases.

From the above background, techniques for measuring kinase activity have been previously developed and used. A commonly used method for measuring the kinase activity is a method of reacting a substrate of protein or peptide with a kinase in a sample and measuring a phosphorylated substrate to obtain the kinase activity. Known means for detecting phosphorylated peptides or proteins include ELISA using anti-phosphorylation antibodies, mobility shift assay, peptide arrays, protein arrays, LC-MS, etc.

### CITATION LIST

### Patent Literature

[PTL 1] JP-A-2009-50171

### SUMMARY OF INVENTION

### Technical Problem

However, the number of phosphorylation sites contained in one molecule of a substrate of protein or peptide varies depending on the type of the substrate, even a reaction of a constant amount of the substrate of protein or peptide with a kinase has a problem that the number of phosphorylation sites actually functioning as a substrate varies depending on the substrate type. The production rate of a phosphorylated amino acid in a substrate of a protein or peptide is affected by the number of phosphorylation sites, and the effect of differences in the number of phosphorylation sites cannot be taken into consideration by conventional kinase activity measurements.

One of the problems to be solved by the embodiments disclosed in this description and the drawings is to enable measurement of enzyme activity taking into consideration the number of phosphorylation sites. However, the problems to be solved by the embodiments disclosed in this description and the drawings are not limited to the above problem. The problems corresponding to the effects of the configurations shown in the embodiments described below can also be regarded as other problems.

### Solution to Problem

The method for measuring kinase activity according to the embodiment includes the steps: (a) making the number of effective phosphorylation sites correspond to a substrate protein; (b) acquiring the amount of the substrate protein on a base; and (c) estimating the kinase activity based on the number of effective phosphorylation sites corresponding to the substrate protein on the base and the acquired amount of the substrate protein on the base.

The apparatus for measuring the kinase activity according to the embodiment is an information processing apparatus that includes the following: an acquisition unit acquiring the amount of a substrate protein on a base; and an estimation unit reading out the number of effective phosphorylation sites corresponding to the substrate protein on the base from a storage unit storing the number of effective phosphorylation sites that are made to correspond to the substrate protein, and estimating the kinase activity based on the number of effective phosphorylation sites and the acquired amount of the substrate protein on the base.

### BRIEF DESCRIPTION OF DRAWINGS

FIG. 1 is a diagram showing an example of a flow of the method for measuring the kinase activity according to the first embodiment.

FIG. 2 is a diagram showing a straight line obtained by measuring the amount of phosphorylated amino acids and the amount of a substrate protein by reacting a kinase with a substrate protein (BCAR1) and plotting the number of phosphorylated amino acids as a function of the amount of the substrate protein on a graph.

FIG. 3 is a diagram showing a straight line obtained by measuring the amount of phosphorylated amino acids and the amount of a substrate protein by reacting a kinase with a substrate protein (Abi1) and plotting the number of phosphorylated amino acids as a function of the amount of the substrate protein on a graph.

FIG. 4 is a diagram showing a straight line obtained by measuring the amount of phosphorylated amino acids and the amount of a substrate protein by reacting a kinase with a substrate protein (Src) and plotting the number of phosphorylated amino acids as a function of the amount of the substrate protein on a graph.

FIG. 5 is a diagram showing a curve obtained by measuring the number of phosphorylated amino acids and the amount of a substrate protein by reacting a kinase with the same activity and a substrate protein with a different number of sites and plotting the number of phosphorylated amino acids as a function of the amount of the substrate protein on a graph.

FIG. 6 is a diagram showing a curve obtained by correcting and normalizing the number of phosphorylated amino acids in the curve obtained in FIG. 5 based on the number of phosphorylation sites obtained.

FIG. 7 is a diagram showing plots for BCAR1 and Src of the average phosphorylation reaction rate with respect to the number of phosphorylation sites obtained, and also the results of calculating Vmax-Km by fitting taking into consideration the initial number of phosphorylation sites.

FIG. 8 is a diagram showing plots for BCAR1 of the amount of phosphorylated amino acids versus the number of phosphorylation sites obtained, and also the results of calculating Vmax-Km by fitting taking into consideration the decrease in the number of phosphorylation sites during the reaction.

### DESCRIPTION OF EMBODIMENTS

Hereinafter, embodiments of the apparatus for measuring kinase activity and the method for measuring the kinase activity will be described in detail with reference to the drawings.

### First Embodiment

The method for measuring the kinase activity according to the first embodiment includes the steps: (a) making the number of effective phosphorylation sites correspond to a substrate protein; (b) acquiring the amount of the substrate protein on a base; and (c) estimating the kinase activity based on the number of effective phosphorylation sites corresponding to the substrate protein on the base and the acquired amount of the substrate protein on the base.

The method for measuring kinase activity according to the first embodiment includes the step of (a) making the number of effective phosphorylation sites correspond to a substrate protein.

The “substrate protein” in the method for measuring the kinase activity according to the first embodiment refers to one or more types of proteins that are phosphorylated by the kinase of activity measurement target, and may be immobilized on a base or contained in a spot. The substrate protein can be appropriately determined depending on the purpose by a person conducting the method for measuring the kinase activity according to the first embodiment, and may be naturally derived, synthetic, or recombinant.

The number of phosphorylation sites (sites subjected to a phosphorylation reaction) in a substrate protein may vary depending on the type of the substrate protein. In the method for measuring the kinase activity according to the first embodiment, the number of phosphorylation sites per molecule of a certain substrate protein is defined as the number of effective phosphorylation sites corresponding to the certain substrate protein. The number of effective phosphorylation sites corresponding to a specific substrate protein is preferably determined for each combination of the substrate protein and kinase.

The method for acquiring the number of effective phosphorylation sites corresponding to a specific substrate protein is not particularly limited, and the number may be acquired directly by calculation, for example, by plotting on a graph the amount of phosphorylated amino acids produced in a labeled phosphorylated substrate protein in a spot as a function of the amount of the substrate protein in the spot and calculating the gradient of the resulting straight line or curve, or by bringing the substrate protein into contact with a kinase for a period of time that enables phosphorylation of all effective phosphorylation sites in the substrate protein and calculating the number of effective phosphorylation sites. The acquired data of the number of effective phosphorylation sites corresponding to a specific substrate protein may be stored and, if necessary, may be used as the value of the number of effective phosphorylation sites corresponding to a specific substrate protein in another measurement.

Furthermore, when the number of effective phosphorylation sites is determined beforehand for a specific substrate protein, this value may be referenced for use. In this case, when the number of effective phosphorylation sites corresponding to a specific substrate protein is publicly known, that value may be referenced for use, for example, a value identified using the PhosphoSitePlus online tool may be referenced for use. In this case, although the number of effective phosphorylation sites corresponding to a specific substrate protein is preferably the value for a kinase of the same type as the measurement target kinase, a value for a kinase of a similar type to the measurement target kinase may also be used as the number of effective phosphorylation sites corresponding to a specific substrate protein.

The method for measuring the kinase activity according to the first embodiment includes the step (b) of acquiring the amount of a substrate protein on a base.

The method for acquiring the amount of a substrate protein is not limited, and examples thereof include quantification by SPR (surface plasmon resonance) method, quantification based on electrochemical properties (e.g., potential, current value, impedance, capacitance, etc.), quantification based on the presence distribution of elements obtained by X-ray spectroscopy etc., quantification using AFM, quantification based on signal information derived from a labeling substance introduced into the substrate, etc. Furthermore, when the amount of the substrate protein is known, it may be used.

The “base” in the method for measuring the kinase activity according to the first embodiment includes, though not limited to, a two-dimensional plate, a polymer gel, a fiber and a fiber sheet, beads, a rod, etc. The surface of the “base” may be smooth, and may also have a micro/nano structure such as a porous structure or fiber. The “base” is preferably a two-dimensional plate, and examples of the two-dimensional plate include plate-like base such as a slide glass and a cover glass, and a well base such as an array plate. The “base” is more preferably a well base such as an array plate. The array plate has spots for containing a substrate and is used for comprehensive analysis of samples, and is also called a microchip, a microarray, a protein chip, a DNA chip, etc.

The “spot” in the method for measuring the kinase activity according to the first embodiment means a fixed closed area on a base. The shape of the “spot” is not particularly limited and may be, for example, a square, a rectangle, a circle, an ellipse, etc., and a circle is preferred. The spot may contain substrate proteins, and in this case, the substrate proteins contained may be immobilized on a base.

The method for measuring the kinase activity according to the first embodiment includes the step of (c) estimating the kinase activity based on the number of effective phosphorylation sites corresponding to the substrate protein on the base and the acquired amount of the substrate protein on the base.

In the method for measuring the kinase activity according to the first embodiment, the “specimen” containing the kinase as a measurement target can be determined appropriately based on the purpose by a person performing the method, and anything containing a kinase can be a specimen. The specimen includes biologically derived substances, extracts from biological bodies, blood, blood-derived substances, food, food-derived substances, natural products, substances derived from natural products, and substances derived from culture medium. The specimen may be pretreated as appropriate depending on the purpose and procedure, or a reagent may be added thereto beforehand. The specimen may be in the form of gas, solid, or liquid, and is appropriately used in liquid form by diluting, suspending, or extracting in water, physiological saline, a buffer solution, or other solution. The specimen may contain preservatives and other additives. In addition, reagents are added to the specimen depending on the purpose.

The method for measuring the kinase activity according to the first embodiment may be a method for measuring the kinase activity of a kinase contained in a specimen using a base having spots containing a substrate.

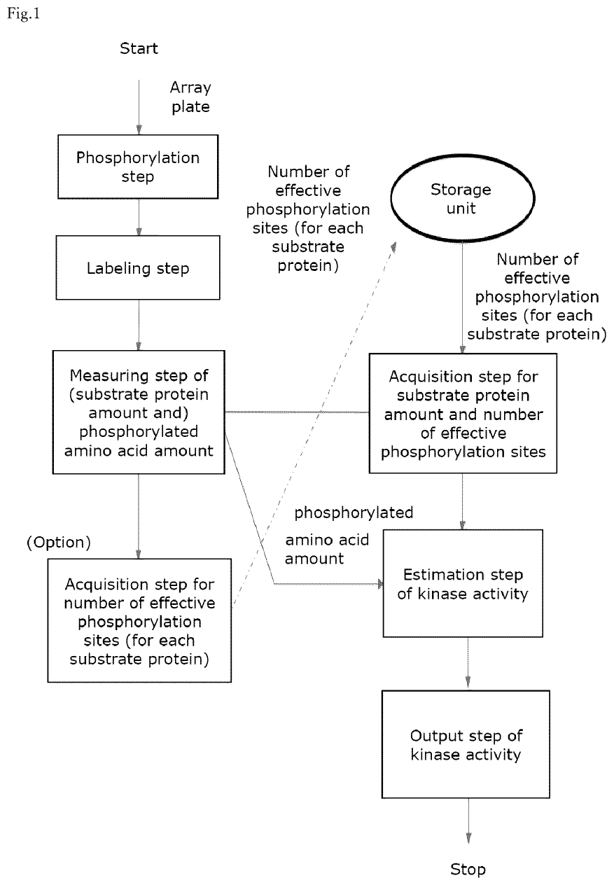

FIG. 1 is a diagram showing an example of a flow of the method for measuring the kinase activity according to the first embodiment. The method for measuring the kinase activity shown in FIG. 1 includes the following steps: a step of performing a phosphorylation reaction between a kinase and a substrate protein to obtain a phosphorylated substrate protein (phosphorylation reaction step); a step of further labeling the phosphorylated substrate protein to obtain a labeled phosphorylated substrate protein (labeling step); a step of measuring the amount of phosphorylated amino acids produced in the labeled phosphorylated substrate protein (and the amount of the substrate protein, if necessary) ((substrate protein amount and) phosphorylated amino acid amount measurement step); a step of acquiring the amount of the substrate protein and the number of effective phosphorylation sites corresponding to the substrate protein (substrate protein amount and the number of effective phosphorylation site acquisition step); a step of estimating a value of the kinase activity of the kinase based on the amount of the substrate protein, the number of effective phosphorylation sites corresponding to the substrate protein, and the amount of phosphorylated amino acids produced in the labeled phosphorylated substrate protein (kinase activity estimation step); and a step of outputting the kinase activity of the kinase based on the estimated value (kinase activity output step).

The method for measuring the kinase activity shown in FIG. 1 includes a step of performing a phosphorylation reaction between a kinase and a substrate protein to obtain a phosphorylated substrate protein. This step may be a step of obtaining the phosphorylated substrate protein by bringing a specimen into contact with a spot on a base and performing a phosphorylation reaction between a kinase contained in the specimen and a substrate protein contained in the spot.

The method for measuring the kinase activity shown in FIG. 1 includes a step of further labeling the phosphorylated substrate protein to obtain a labeled phosphorylated substrate protein. Further labeling the phosphorylated substrate protein means introducing a labeling substance into the phosphorylated substrate protein via a phosphorylation site recognition substance. The phosphorylation site recognition substance refers to a substance recognizing specifically a phosphorylated site in a substrate protein phosphorylated by a phosphorylation enzyme. The phosphorylation site recognition substance is not particularly limited, and examples thereof include anti-phosphorylation amino acid antibodies. When a labeling substance is not bonded to the phosphorylation site recognition substance, the labeling substance may be further introduced via a specific reaction including a secondary antibody or a biotin-avidin reaction.

Examples of labeling substances include radioactive substances, enzymes, capture molecules, fluorescent substances, luminescent substances, metal particles, etc., and are preferably optically detectable labeling substances, which examples include fluorescent substances, chemiluminescent substances, phosphorescent substances, dyes, gold nanoparticles, fluorescent particles, enzymes for enzymatic chemiluminescence or color reactions, and microparticles absorbing at specific wavelength. The labeling substance may also include an antibody, ligand, or other binding site. The method for binding or introducing the labeling substance may include binding the labeling substance via hydrophobic interaction, electrostatic interaction, van der Waals interaction, hydrogen bonding, or covalent bonding; introducing during synthesis, etc., and the labeling methods include those publicly known.

The method for measuring the kinase activity shown in FIG. 1 includes a step of acquiring the amount of the phosphorylated amino acid produced in a labeled phosphorylated substrate protein, and, if necessary, the amount of the substrate protein. The amount of the phosphorylated amino acid produced in the labeled phosphorylated substrate protein and the substrate protein may be measured as amounts in the spot.

The amount of the substrate protein can be measured by known methods, such as quantification by SPR (surface plasmon resonance) method, quantification based on electrochemical properties (e.g., potential, current value, impedance, capacitance, etc.), quantification based on the presence distribution of elements obtained by X-ray spectroscopy etc., quantification using AFM, quantification based on signal information derived from a labeling substance introduced into the substrate, etc. Furthermore, when the amount of the substrate protein is known, it may be used.

The amount of the phosphorylated amino acid produced in the labeled phosphorylated substrate protein is quantified based on the signal information derived from the labeling substance. The intensity of the signal provides information on the amount of the labeled phosphorylated substrate protein, which enables quantification of the amount of phosphorylated amino acids produced in the labeled phosphorylated substrate protein. Such a signal is preferably optically detectable, and examples thereof include light intensity information, spectroscopic information, etc. from the spot.

When the labeling substance is a fluorescent substance, any optical system can be used as long as being capable of exciting the fluorescent substance and detecting the fluorescence. An excitation light source can be used to excite the fluorescent substance, and examples thereof include a laser light source, a light emitting diode, an LED, a mercury arc, and a tungsten halogen lamp. For detection, a CCD camera, a photodiode, etc. may be used. The optical system appropriately includes filters to irradiate or detect light of limited wavelengths. The optical system may also include a lens. The optical system may be of the scanning or non-scanning type. Specifically, a confocal optical unit can be used in the optical system.

When chemiluminescence, dyes, etc. are used as the labeling substance, an excitation light source is not necessary.

The method for measuring the kinase activity shown in FIG. 1 includes a step of acquiring the amount of a substrate protein and the number of effective phosphorylation sites corresponding to the substrate protein. The amount of the substrate protein and the number of effective phosphorylation sites corresponding to the substrate protein may be those in the spot.

The method for measuring the kinase activity shown in FIG. 1 includes a step of estimating the value of the kinase activity of the kinase based on the amount of a substrate protein, the number of effective phosphorylation sites corresponding to the substrate protein, and the amount of phosphorylated amino acids produced in the labeled phosphorylated substrate protein.

The estimation of value of the kinase activity in this step is performed by correcting the amount of phosphorylated amino acids produced in the measured labeled phosphorylated substrate protein based on the number of effective phosphorylation sites corresponding to the substrate protein, and, for example, the amount of phosphorylated amino acids produced in the measured labeled phosphorylated substrate protein may be simply normalized by the determined number of effective phosphorylation sites corresponding to the substrate protein, and preferably by taking into consideration the decrease, as the phosphorylation reaction proceeds, in the number of effective phosphorylation sites corresponding to the substrate protein.

The estimation of the kinase activity value taking into consideration the decrease, as the phosphorylation reaction progresses, in the number of effective phosphorylation sites corresponding to the substrate protein can be performed, for example, by the following: (i) acquiring, at each point of the spot, the amount of the substrate protein at the beginning of the reaction and the amount of phosphorylated amino acids produced in the phosphorylated substrate protein after a specified reaction time has elapsed; (ii) converting the amount of the substrate protein at the beginning of the reaction into the number of effective phosphorylation sites corresponding to the substrate protein; (iii) by assuming certain Km and Vmax and using a phosphorylation production rate determined based on the number of effective phosphorylation sites corresponding to the substrate protein at the beginning of the reaction, yielding the production of phosphorylated amino acids and the decrease in the number of effective phosphorylation sites corresponding to the substrate protein after a unit time has elapsed; (iv) determining the phosphorylation production rate in the next unit time based on the decreased number of effective phosphorylation sites corresponding to the substrate protein; (v) repeating (iii) and (iv) to obtain an estimated result of the relationship between the amount of the initial substrate protein and the amount of phosphorylated amino acids produced when a specified reaction time has elapsed; and (vi) converging Vmax and Km so that the error between the estimated result obtained in (v) and the actual measurement result is minimized. A method for measuring the kinase activity that includes this step enables elimination of bias due to difference in the number of effective phosphorylation sites corresponding to substrate proteins, and more accurate measurement of the activity of the measurement target kinase.

The method for measuring kinase activity shown in FIG. 1 includes a step of outputting the kinase activity of the kinase based on the estimated value.

### Second Embodiment

The apparatus for measuring the kinase activity according to the second embodiment is an information processing apparatus that includes the following: an acquisition unit acquiring the amount of a substrate protein on a base; and an estimation unit reading out the number of effective phosphorylation sites corresponding to the substrate protein on the base from a storage unit storing the number of effective phosphorylation sites that is made to correspond to the substrate protein, and estimating the kinase activity based on the number of effective phosphorylation sites and the acquired amount of the substrate protein on the base. The storage unit may be further provided in the information processing apparatus, or may be connected to the information processing apparatus via a network.

The meanings and references of the terms “substrate protein,” “base,” “number of effective phosphorylation sites,” etc. used to explain the second embodiment are the same as those used to explain the first embodiment.

The amount of a substrate protein on the base may be acquired as a result of the actual measurement of the amount of the substrate protein by the acquisition unit of the apparatus for measuring the kinase activity of the second embodiment and if the amount of the substrate protein is publicly known, that value may be acquired as the amount of the substrate protein on the base.

At least one of the embodiments described above enables measurement of the kinase activity taking into consideration the number of phosphorylation sites.

Although several embodiments have been described, these embodiments are presented as examples and are not intended to limit the scope of the invention. These embodiments can be implemented in various other forms, and various omissions, substitutions, modifications, and combinations of embodiments can be made without departing from the spirit of the invention. These embodiments and their variations are included in the scope equivalent to that of the invention described in the scope of claims as well as in the scope and spirit of the invention.

### EXAMPLES

The present invention will be specifically described based on the following examples, but the present invention is not limited to these examples. Unless otherwise specified, the contents are expressed in mass %.

### Example 1. Estimation of the Number of Phosphorylation Sites of Each Substrate Protein

Three substrate proteins, namely, GST (glutathione-S-transferase) tagged BCAR1, GST tagged Abi1, and GST tagged Src, were selected, and exemplified was estimation of the number of phosphorylation sites of the substrate proteins for a group of kinases contained in a cell extract of the human leukemia cell line K562. Generally, there are three types of amino acids that are phosphorylated: serine, threonine, and tyrosine, and here exemplified was estimation of the number of phosphorylation sites in each substrate with respect to phosphorylation of tyrosine.

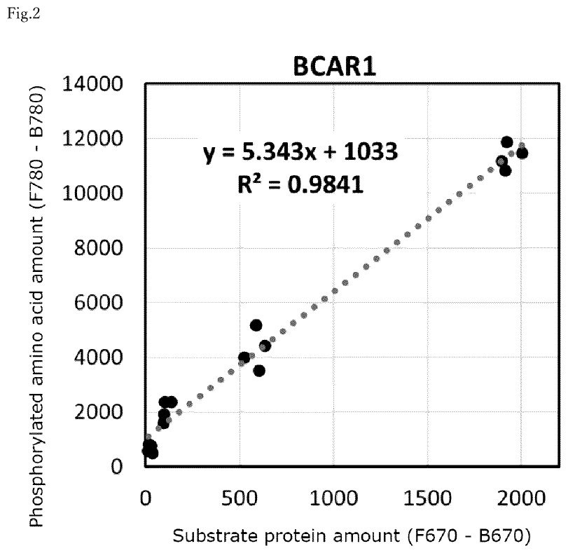

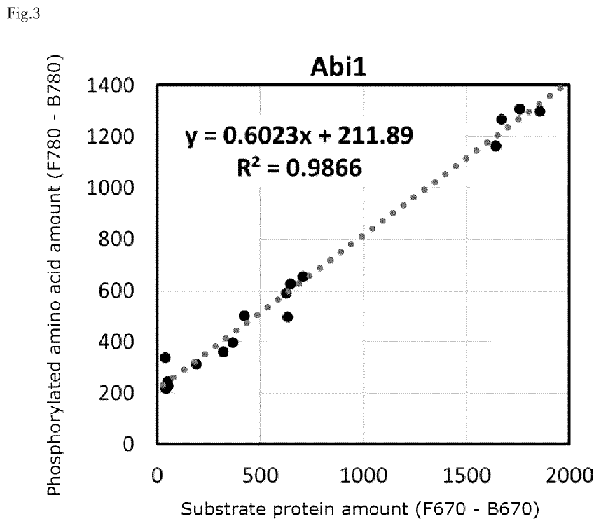

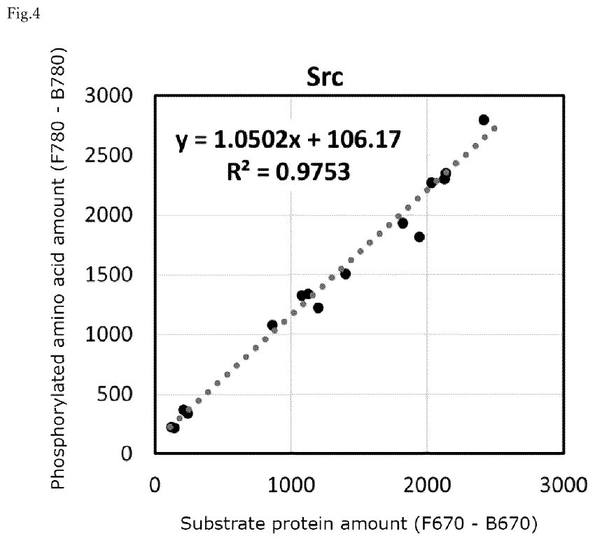

For each substrate protein, prepared was a dilution series of spotting solutions with the protein concentration ratios of 1, 2, 4, and 8. The above spotting solutions were dropped onto a glutathione (GSH)-coated slide glass prepared by the method described in non-patent literature (Tadashi Manabe et al., “IGF2 Autocrine-Mediated IGF1R Activation Is a Clinically Relevant Mechanism of Osimertinib Resistance in Lung Cancer”, Mol Cancer Res. 2020 April; 18 (4): 549-559.), at 4 spots for each concentration, to obtain an array plate having spots (diameter approximately 100 μm) of immobilized GST-tagged BCAR1, Abi1, and Src. In addition, an excess amount (about 100 μg) of cell extract from human leukemia cell line K562 was mixed with a kinase reaction solution (25 mM Tris-HCl, 5 mM β-glycerophosphate, 0.1 mM Na3VO4, 10 mM MgCl2, 1 mM ATP, and 2 mM DTT), brought into contact with the immobilization spots of each protein, and incubated at 30° C. for a sufficiently long time (about 2 hours), thereby inducing a phosphorylation reaction at all phosphorylation sites, effective against the kinase contained in the K562 cell extract, among phosphorylation sites in each substrate protein. The reaction was then stopped by bringing a reaction stop solution (50 mM EDTA, 10 mM HEPES-NaOH [pH 7.4], 150 mM NaCl, and 0.05% [v/v] Tween 20) into contact with the immobilized spots and incubating at 30° C. for 5 minutes. After washing the array plate with TBST, a primary antibody reaction solution (a cocktail of mouse anti-phosphotyrosine antibody and rabbit anti-GST antibody) was added and incubated at 30° C. for 1 hour. After washing the array plate with TBST, secondary antibody solution (a cocktail of Goat anti-Mouse IgG (H+L) Highly Cross-Adsorbed Secondary Antibody, Alexa Fluor 790 (Invitrogen) and Goat anti-Rabbit IgG (H+L) Highly Cross-Adsorbed Secondary Antibody, Alexa Fluor 680 (Invitrogen)) was added and incubated at 30° C. for 1 hour, and thus the phosphorylated tyrosine generated in the immobilized spots of BCAR1, Abi1, and Src was labeled with Alexa Fluor 790, and the GST tag in BCAR1, Abi1, and Src was labeled with Alexa Fluor 680. The labeled array plate was measured using a microarray scanner having a confocal optical system to obtain a fluorescent image. For Alexa Fluor 790, an excitation laser with a wavelength of 780 nm was used for measurement, and for Alexa Fluor 680, an excitation laser with a wavelength of 670 nm was used for measurement. For each substrate protein, the fluorescence intensity indicating the amount of phosphorylated tyrosine per spot (F780-B780: F780 is the average fluorescence intensity per spot, and B780 is the average intensity of the area outside the spot) was plotted as a function of the fluorescence intensity indicating the amount of the substrate per spot (F670-B670: F670 is the average fluorescence intensity per spot, and B670 is the average intensity of the area outside the spot) (FIGS. 2, 3, and 4). Under the conditions used in this example, all effective phosphorylation sites were phosphorylated, and therefore the spot brightness indicating the amount of phosphorylated tyrosine for each protein showed a straight line with a constant slope with respect to the spot brightness indicating the amount of the substrate. This slope indicates the amount of phosphorylated tyrosine obtained per unit amount of the substrate protein, further under the condition of saturated phosphorylation reaction, the amount of phosphorylated tyrosine indicates the number of effective phosphorylation sites, and thus by comparing the slopes among the three species of BCAR1, Abi1, and Src, the ratio of the number of effective phosphorylation sites was obtained. The ratio of slopes for BCAR1:Abi1:Src is as follows:
5.34:0.602:1.05≃5:0.5:1,
   and this was the ratio of the number of effective phosphorylation sites for BCAR1, Abi1, and Src when phosphorylation reaction was performed with a K562 cell extract. To determine the number of phosphorylation sites from the ratio f the number of phosphorylation site, the number of phosphorylation sites reported for BCAR1 was sought by referring to PhosphoSitePlus, a known database. PhosphoSitePlus contains information on phosphorylation sites for each substrate protein, obtained primarily by techniques such as LC-MS for various samples. In this example, among tyrosine residues in BCAR1 for which phosphorylation has been reported, those residues for which 50 or more reports exist were extracted, thereby identifying 12 potential phosphorylation sites. The above slope ratio of BCAR1:Abi1:Src is 5:0.5:1, and considering that the number of phosphorylation sites is an integer, the ratio of the number of phosphorylation sites is also possibly 10:1:2=20:2:4=30:3:6; however, since the potential maximum number of phosphorylation sites is 12 and thus does not exceed 12, the numbers of effective phosphorylation sites of BCAR1, Abi1, and Src for kinases contained in the K562 cell extract were estimated to be 10, 1, and 2, respectively.

In measuring the activity of the kinase group contained in K562, the number of effective phosphorylation sites was estimated by multiplying the amount of the substrate measured for each substrate spot by the number of effective phosphorylation sites obtained above.

### Example 2. Normalization of Phosphorylated Amino Acid Amount by Number of Phosphorylation Sites

Normalization of the amount of phosphorylated amino acid by the number of phosphorylation sites is exemplified in the case of measuring the amount of the substrate protein and the phosphorylated amino acid per spot for determination of the kinase activity by bringing a reaction solution containing a kinase into contact with an array plate having spots of substrate proteins to induce a phosphorylation reaction.

This example was conducted, in the same manner as in Example 1, by preparing an array plate having spots of substrate proteins, inducing phosphorylation reaction by kinase, labeling, and measuring the amounts of substrate proteins and phosphorylated amino acids. However, determination of the kinase activity required phosphorylation at only a portion of the phosphorylation sites (i.e., not conditions of all phosphorylation sites phosphorylated or saturated phosphorylation) and thus to prevent the phosphorylation sites from entire phosphorylation, the reaction conditions were controlled by reducing the amount of cell extract or shortening the phosphorylation reaction time.

The amount of substrate per spot was multiplied by the number of phosphorylation sites of the substrate protein, and converted into the number of phosphorylation sites per spot. The phosphorylation reaction rate amount of phosphorylated amino acid per spot was considered to be the average phosphorylation reaction rate over the predetermined phosphorylation reaction time. Under phosphorylation reaction conditions of small number of phosphorylation sites, the phosphorylation reaction rate is limited by the number of phosphorylation sites, making it difficult to use only the amount of phosphorylated amino acids per spot as an index of the kinase activity. Therefore, the amount of phosphorylated amino acids was normalized by dividing the amount of phosphorylated amino acids per spot by the number of phosphorylation sites per spot. Normalizing was performed to correct the effect of the reaction rate limiting due to the number of phosphorylation sites, and the value of the kinase activity was compared.

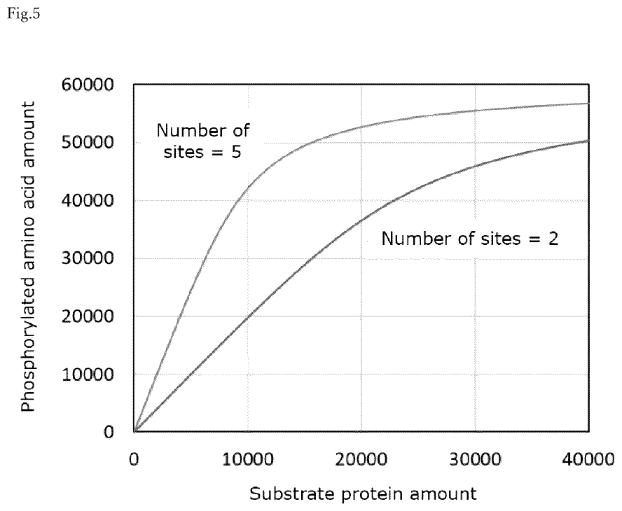

An example of the correction is shown in FIG. 5. Here, an example of phosphorylation reaction is as follows: in accordance with the Michaelis-Menten equation:

$$v = \frac{{V}_{max} [ S ]}{[ S ] + {K}_{m}} , \qquad [ Math . 1 ]$$

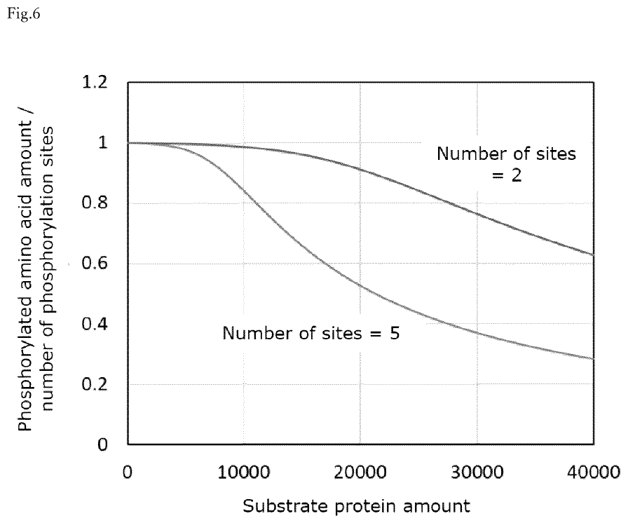

assuming a phosphorylation reaction with the kinase activity of Km=10000 and Vmax=1000, the relationship between the amount of phosphorylated amino acids produced in a 60-minute phosphorylation reaction with substrate amounts of 0 to 40000 is shown for the cases of the number of effective substrate sites of 2 and 5. Since the kinase activity is the same for the cases of the number of phosphorylation sites of 2 and 5, the return of indicator of the kinase activity is desirably the same value regardless of the number of phosphorylation sites. When the amount of the substrate protein is small and in the range where the amount of the substrate protein is rate-limiting for the phosphorylation reaction (≤5000), the produced amount of phosphorylated amino acids for 5 phosphorylation sites is approximately 2.5 times higher than that for 2 phosphorylation sites, resulting in a large error for the case of using the amount of phosphorylated amino acids as an index of the kinase activity. FIG. 6 shows the results of normalizing FIG. 5 based on the number of phosphorylation sites. Use of the normalized amount of phosphorylated amino acids as an index of the kinase activity enables recognition that the kinase activities for the number of phosphorylation sites of 2 and 5 was the same when the amount of the substrate protein was small and in the range where the amount of the substrate protein is rate-limiting for the phosphorylation reaction (≤5000).

### Example 3. Estimation of Vmax-Km by Fitting Using the Michaelis-Menten Equation

Estimation of the maximum phosphorylation reaction rate Vmax and the Michaelis constant Km by fitting the number of phosphorylation sites at the beginning of the reaction and the distribution of the amount of phosphorylated amino acid using the Michaelis-Menten equation is exemplified in the case of measuring the amount of the substrate protein and phosphorylated amino acid per spot for determination of the kinase activity by bringing a reaction solution containing a kinase into contact with an array plate having spots of substrate proteins to induce a phosphorylation reaction.

This example was conducted, in the same manner as in Example 1, by preparing an array plate having spots of substrate proteins, inducing phosphorylation reaction by kinase, labeling, and measuring the amounts of substrate proteins and phosphorylated amino acids. However, determination of the kinase activity required phosphorylation at only a portion of the phosphorylation sites (i.e., not conditions of all phosphorylation sites phosphorylated or saturated phosphorylation) and thus to prevent the phosphorylation sites from entire phosphorylation, the reaction conditions were controlled by reducing the amount of cell extract or shortening the phosphorylation reaction time.

Based on the results of measuring the amounts of the substrate protein and phosphorylated amino acids, the distribution of the amount of phosphorylated amino acids with respect to the amount of the substrate protein was plotted two-dimensionally. A Plot of the amount of phosphorylated amino acids with respect to different amounts of the substrate protein was necessary to perform fitting using the Michaelis-Menten equation, which was obtained by preparing spots using spot solutions having different substrate protein concentrations, as in Example 1, and plotting the amount of phosphorylated amino acids per spot with respect to the amount of the substrate protein per spot. Alternatively, when variation in the amount of immobilization of the substrate protein was observed at each point within a spot prepared using a spot solution having a certain substrate protein concentration, the amount of the substrate protein and the amount of phosphorylated amino acids at each point were plotted to obtain the plot.

For the obtained plot, the amount of the substrate protein was multiplied by the number of phosphorylation sites of that substrate protein, and the amount of phosphorylated amino acids was divided by the phosphorylation reaction time to be converted into an average phosphorylated amino acid production rate, thereby yielding a distribution of phosphorylated amino acid production rates with respect to the number of phosphorylation sites. The obtained distribution is fitted with the Michaelis-Menten equation:

$$v = \frac{{V}_{max} ⌈ S ⌉}{⌈ S ⌉ + {K}_{m}} . \qquad [ Math . 2 ]$$

Here, [S] is the number of phosphorylation sites, v is the production rate of phosphorylated amino acid, and Vmax and Km were obtained as indices of the kinase activity.

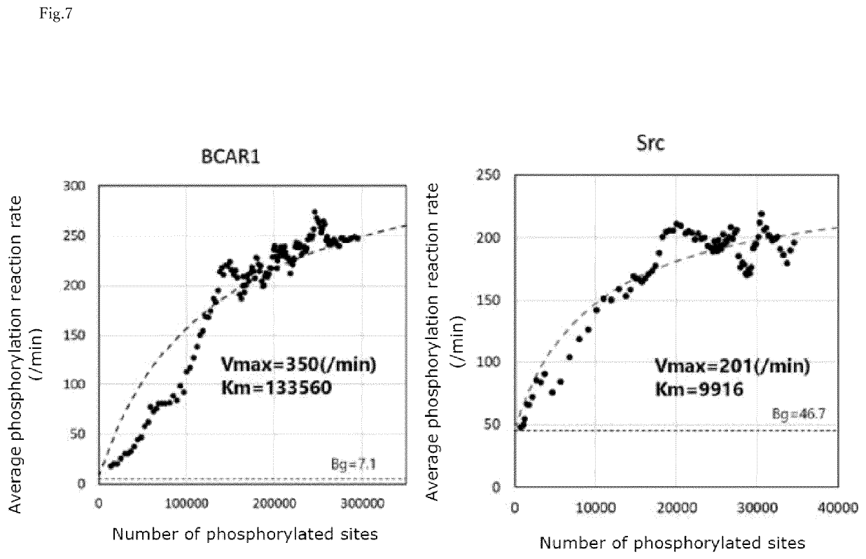

For the BCAR1 and Src spots, phosphorylation reaction was performed for 120 minutes with 10 μg of K562 cell extract to determine the amounts of the substrate protein and phosphorylated amino acids by plotting the fluorescence brightness indicating the amounts of the substrate protein and phosphorylated amino acids at each point in the spot, then the numbers of phosphorylated amino acid (tyrosine) sites of each protein type were set to 10 and 2 (the values determined in Example 1), respectively, and a graph converted to the relationship between the number of phosphorylated amino acid sites and the average reaction rate obtained by dividing the amount of phosphorylated amino acid by the phosphorylation reaction time (120 minutes) was subjected to fitting by the method of Example 3, which results are shown in FIG. 7. The conditions for the phosphorylation reaction was changed to reduce the amount of lysate compared to Example 1. Upon changing the conditions, it was observed that the amount of phosphorylated amino acids is nonlinearly correlated with the number of phosphorylation sites, suggesting that only a portion of the phosphorylation sites is actually phosphorylated (phosphorylation was not saturated). The results of fitting were Km=133560 and Vmax=350 (/min) for BCAR1, and Km=9916 and Vmax=201 (/min) for Src. Consideration of the difference in the number of phosphorylation sites enables the difference in affinity between BCAR1 and Src to be expressed as a difference in Km, and suggestion that Src has a smaller Km and higher affinity with the kinase group contained in the K562 cell extract. Incidentally, the fitting was performed, due to a background noise of fluorescence brightness (F780-B780) indicating the amount of phosphorylated amino acids, by setting the background of the average phosphorylation reaction rate with 7.1 for BCAR1 and 46.7 for Src.

Adopting the Michaelis-Menten equation to the distribution of the amount of phosphorylated amino acids with respect to the number of phosphorylation sites conceivably enables more accurate estimation of Vmax as the kinase activity in this example than that in Example 2. The Km determined by the fitting for 120-minute phosphorylation reaction in this example indicated the number of phosphorylation sites at the initial stage of the reaction possibly corresponding to the average phosphorylation reaction rate at a half of the maximum reaction rate Vmax.

### Example 4. Estimation of Vmax-Km by the Michaelis-Menten Equation Taking into Consideration the Decrease in the Number of Phosphorylation Sites in the Middle of the Reaction

Estimation of the maximum phosphorylation reaction rate Vmax and the Michaelis constant Km by fitting with the Michaelis-Menten equation, while taking into consideration the number of phosphorylation sites at the beginning of the reaction and the decrease in the number of unreacted phosphorylation sites during the reaction, is exemplified in the case of measuring the amount of the substrate protein and phosphorylated amino acid per spot for determination of the kinase activity by bringing a reaction solution containing a kinase into contact with an array plate having spots of substrate proteins to induce a phosphorylation reaction.

This example was conducted, in the same manner as in Example 1, by preparing an array plate having spots of substrate proteins, inducing phosphorylation reaction by a kinase, labeling, and measuring the amounts of substrate proteins and phosphorylated amino acids. However, determination of the kinase activity required phosphorylation at only a portion of the phosphorylation sites (i.e., not conditions of all phosphorylation sites phosphorylated or saturated phosphorylation) and thus to prevent the phosphorylation sites from entire phosphorylation, the reaction conditions were controlled by reducing the amount of cell extract or shortening the phosphorylation reaction time.

As in Example 3, the plot, showing the distribution of the amount of phosphorylated amino acids with respect to the amount of the substrate protein, was obtained and the plot, showing the distribution of the amount of phosphorylated amino acids with respect to the number of phosphorylation sites, was obtained by multiplying the amount of the substrate protein by the number of phosphorylation sites of the substrate protein. Here, since the amount of the substrate protein was determined using the fluorescent intensity from GST tag which is fused to a substrate protein and fluorescently labeled, the number of phosphorylation sites indicated the number present at the beginning of the reaction. The number of unreacted phosphorylation sites decreased as the phosphorylation reaction progresses. Taking the above into consideration, fitting with the Michaelis-Menten equation was performed. When assuming a certain Km and Vmax, the production rate of phosphorylated amino acids and the decrease in the number of unreacted phosphorylation sites after a unit time were determined based on the number of unreacted phosphorylation sites at the beginning of the reaction. Sequentially, based on the decreased number of phosphorylation sites, the phosphorylation production rate in the next unit time was determined. This procedure was repeated until the phosphorylation reaction time was reached, and the correspondence between the number of phosphorylation sites and the amount of phosphorylated amino acids was estimated. Vmax and Km were converged so that the error was minimized between the correspondence estimated here and the distribution of the actually measured amount of phosphorylated amino acids and the number of phosphorylation sites, to obtain the resulting Vmax and Km. This example, compared to Example 3, by calculating the phosphorylation reaction rate at every unit time taking into consideration the effect of a decrease in the reaction rate due to a decrease in the number of unreacted substrate sites, and thus eliminating a bias due to differences in the number of effective phosphorylation sites in the substrate, enabled more accurate measurement of the activity of the measurement target kinase.

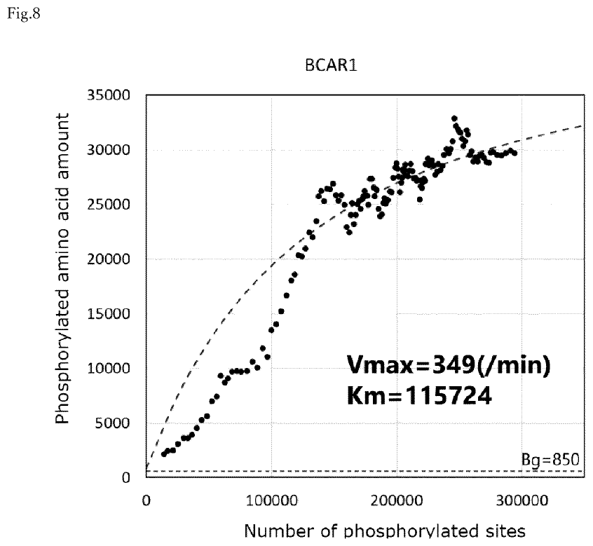

The results of fitting performed by the method of Example 4 on the graph showing the relationship between the number of phosphorylated amino acid sites and the amount of phosphorylated amino acids in the BCAR1 spots obtained in Example 3 are shown in FIG. 8. As shown in FIG. 8, Vmax=349 (/min) and Km=115724 were obtained. The Km value was smaller than that in Example 3, which suggests that the fitting performed taking into consideration the decrease in the number of unreacted phosphorylation sites prevented an overestimation of the Km, enabling estimation of the Km value closer to the true value.

Incidentally, although the Km and Vmax values obtained in Examples 2 to 4 are in arbitrary units due to their origin of measured values of fluorescence brightness, conversion of the units is possible, for example, by preparing calibration curves based on the relationship between the known amount of phosphorylated amino acid and fluorescence brightness, and the known number of phosphorylation sites and fluorescence brightness.

1. A method for measuring kinase activity comprising:
(a) making the number of effective phosphorylation sites correspond to a substrate protein;
(b) acquiring the amount of the substrate protein on a base; and
(c) estimating the kinase activity based on the number of effective phosphorylation sites corresponding to the substrate protein on the base and the acquired amount of the substrate protein on the base.

2. The method according to claim 1, wherein step (a) includes a step of calculating the number of effective phosphorylation sites by bringing the substrate protein into contact with a kinase for a period of time that enables phosphorylation of all effective phosphorylation sites in the substrate protein, and making the calculated number of effective phosphorylation sites correspond to the substrate protein.

3. The method according to claim 1, wherein step (a) includes a step of referring to a value determined beforehand as the number of effective phosphorylation sites for the substrate protein and making the referred number of effective phosphorylation sites correspond to the substrate protein.

4. The method according to claim 1, wherein the number of effective phosphorylation sites is made to correspond to a substrate protein for each combination of a substrate protein and a kinase.

5. An information processing apparatus comprising:
an acquisition unit for acquiring the amount of a substrate protein on a base; and
an estimation unit reading out the number of effective phosphorylation sites corresponding to the substrate protein on the base from a storage unit storing the number of effective phosphorylation sites that is made to correspond to the substrate protein, and estimating kinase activity based on the number of effective phosphorylation sites and the acquired amount of the substrate protein on the base.

6. The information processing apparatus according to claim 5, further comprising the storage unit.

7. The information processing apparatus according to claim 5, wherein the storage unit is connected to the information processing apparatus via a network.
